SEQUENCE LISTING (2 sequences)

SEQ ID NO: 1 - DNA, 21 bp, Artificial Sequence
  Primer
caatggtttaacaggcacagg

SEQ ID NO: 2 - DNA, 21 bp, Artificial Sequence
  Primer
ctcaagtgtctgtggatcacg

(19) United States
(12) Patent Application Publication
(10) Pub. No.: US 2021/0220377 A1
(43) Pub. Date: Jul. 22, 2021

(54) USE OF SUBSTITUTED AMINOPROPIONATE COMPOUNDS IN TREATMENT OF SARS-COV-2 INFECTION

(71) Applicant: Academy of Military Medical Sciences, Beijing (CN)

(72) Inventors: Wu ZHONG, Beijing (CN); Ruiyuan Cao, Beijing (CN); Gengfu Xiao, Beijing (CN); Zhihong Hu, Beijing (CN); Manli Wang, Beijing (CN); Leike Zhang, Beijing (CN); Wei Li, Beijing (CN); Yuexiang Li, Beijing (CN); Lei Zhao, Beijing (CN); Shiyong Fan, Beijing (CN); Song Li, Beijing (CN)

(73) Assignee: Academy of Military Medical Sciences, Beijing (CN)

(21) Appl. No.: 17/070,271

(22) Filed: Oct. 14, 2020

(30) Foreign Application Priority Data
Jan. 21, 2020 (CN) 202010071087.7

(51) Int. Cl. A61K 31/685; A61P 31/14

(52) U.S. Cl. CPC A61K 31/685; A61P 31/14

(57) ABSTRACT

The present application relates to use of a substituted aminopropionate compound represented by Formula I, a geometric isomer, a pharmaceutically acceptable salt, a solvate and/or a hydrate thereof, and a pharmaceutical composition comprising the compound for the treatment of a disease or an infection caused by a SARS-CoV-2.

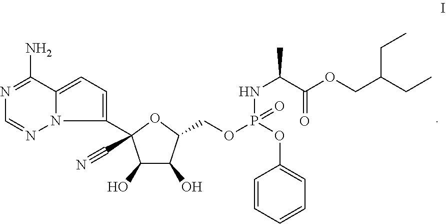 [SMILES: CCC(CC)COC(=O)[C@H](C)NP(=O)(OC[C@H]1O[C@@](C#N)(c2ccc3c(N)ncnn23)[C@H](O)[C@@H]1O)Oc1ccccc1]

The present application is based on and claims the benefit of priority from Chinese application No. 202010071087.7, filed on Jan. 21, 2020, the disclosure of which is incorporated herein by reference in its entirety.

### REFERENCE TO SEQUENCE LISTING SUBMITTED ELECTRONICALLY

The content of the electronically submitted substitute sequence listing, file name 2020-10-13_SeqListing_3274-0280001.txt, size 665 bytes; and date of creation Oct. 13, 2020, filed herewith, is incorporated herein by reference in its entirety.

### TECHNICAL FIELD

The present application relates to use of a substituted aminopropionate compound represented by the following Formula I, a geometric isomer, a pharmaceutically acceptable salt, a solvate and/or a hydrate thereof, and a pharmaceutical composition comprising the above compound in treating a SARS-CoV-2 infection.

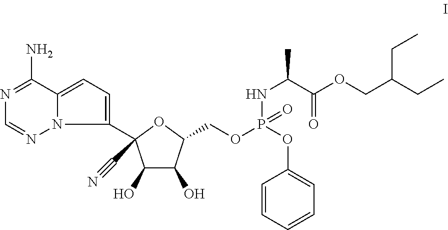 [SMILES: CCC(CC)COC(=O)[C@H](C)NP(=O)(OC[C@H]1O[C@@](C#N)(c2ccc3c(N)ncnn23)[C@H](O)[C@@H]1O)Oc1ccccc1]

### BACKGROUND ART

The substituted aminopropionate compound (Compound represented by Formula I), with chemical name of 2-ethylbutyl (2S)-2-[[[(2R,3 S,4R,5R)-5-(4-aminopyrrolo[2,1-f][1,2,4]triazin-7-yl)-5-cyano-3,4-dihydroxyoxolan-2-yl]methoxy-phenoxyphosphoryl]amino]propanoate, also known as Remdesivir (GS-5734), is a viral RNA polymerase inhibitor, and its active form in vivo is a nucleoside triphosphate (NTP) of the parent drug. The phase III clinical trial of the drug compound for clinical treatment of Ebola virus infection has been now completed, and has shown good therapeutic effect on Ebola virus infection.

Remdesivir has shown antiviral activity against various variants of EBOV and other filoviruses in cell-based assays. In the Ebola virus-infected rhesus monkey model, even if the treatment started three days after virus exposure (systemic viral RNA was detected in two of the six treated animals), the intravenous administration of 10 mg/kg of GS-5734 per day for consecutive 12 days could significantly inhibit the replication of EBOV, protect 100% EBOV-infected animals from death, and improve clinical signs and pathophysiological markers. In addition to Ebola virus, GS-5734 also has a broad-spectrum antiviral activity to viruses such as Nipah virus, Middle East Respiratory Syndrome Coronavirus (MERS-CoV), Marburg virus, etc.

The 2019 novel Coronavirus (2019-nCoV) is a new coronavirus strain that has never been found in humans before. On Feb. 11, 2020, the International Committee on Taxonomy Viruses (ICTV) announced that the official name of 2019 novel Coronavirus (2019-nCoV) is severe acute respiratory syndrome coronavirus 2 (SARS-CoV-2). On the same day, the World Health Organization (WHO) announced that the official name of the disease caused by this virus is COVID-19. The symptoms of SARS-CoV-2 virus infection are mainly pneumonia, and can be divided into simple infection, mild pneumonia, severe pneumonia, acute respiratory distress syndrome, sepsis, septic shock and so on according to the severity of disease. Patients with simple infection may have non-specific symptoms, such as fever, cough, sore throat, nasal congestion, fatigue, headache, muscle pain or discomfort, and the elderly people and immunosuppressed people may have atypical symptoms. Patients with mild pneumonia mainly have cough, dyspnea and polypnea. Severe pneumonia can be seen in adolescents, adults or children, and the main symptoms of which include increased breathing frequency, severe respiratory failure or dyspnea, central cyanosis, drowsiness, unconsciousness or convulsion, gasp, etc. The lung images of acute respiratory distress syndrome are bilateral ground glass shadows, which cannot be completely explained by effusion, lobar exudation or atelectasis or lung mass shadows, and the main symptom of which is pulmonary edema. Patients with sepsis often have fatal organ dysfunction, and the most critical patients are those with septic shock, and they may have a high probability of death.

At present, the SARS-CoV-2 virus infection is mainly treated with supportive therapy in clinic, and no antiviral drug is available.

### Contents of the Application

The purpose of the present application is to find a drug with antiviral activity against SARS-CoV-2, which can be used for the treatment of a related disease caused by the infection thereof. Through creative research, it is found in the present application that the compound represented by Formula I has the function of inhibiting the replication of SARS-CoV-2 and has a good potential therapeutic effect in the treatment of a disease caused by SARS-CoV-2.

The present application relates to the compound represented by Formula I, a geometric isomer, a pharmaceutically acceptable salt and/or a solvate or a hydrate thereof:

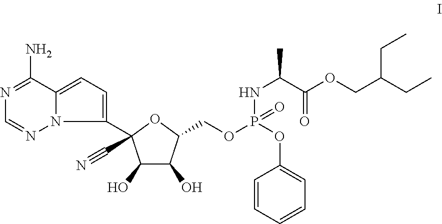 [SMILES: CCC(CC)COC(=O)[C@H](C)NP(=O)(OC[C@H]1O[C@@](C#N)(c2ccc3c(N)ncnn23)[C@H](O)[C@@H]1O)Oc1ccccc1]

In some embodiments, the pharmaceutically acceptable salts of the compound represented by Formula I described herein include inorganic or organic acid salts and inorganic or organic base salts thereof. The present application relates to all forms of the above salts, including but not limited to: sodium salt, potassium salt, calcium salt, lithium salt, meglumine salt, hydrochloride salt, hydrobromide salt, hydroiodide salt, nitrate salt, sulfate, hydrogen sulfate, phosphate, hydrogen phosphate, acetate, propionate, butyrate, oxalate, pivalate, adipate, alginate, lactate, citrate, tartrate, succinate, maleate, fumarate, picrate, aspartate, gluconate, benzoate, methanesulfonate, ethanesulfonate, benzenesulfonate, p-toluenesulfonate and embonate and so on.

The compound represented by Formula I can inhibit SARS-CoV-2 virus replication in cells and reduce the nucleic acid load of SARS-CoV-2 virus in cell culture.

After creative invention research, the inventors of the present application have discovered new features of the compound represented by Formula I in cells: the compound represented by Formula I can reduce the viral nucleic acid load in cells infected by SARS-CoV-2 at micromolar concentration level.

The present application also relates to use of the compound represented by Formula I, a geometric isomer, a pharmaceutically acceptable salt, a solvate and/or a hydrate thereof in manufacture of a medicament for treating a disease or an infection (e.g., a respiratory disease (e.g., a simple infection (such as fever, cough and sore throat), pneumonia, acute respiratory infection, severe acute respiratory infection (SARI), hypoxic respiratory failure and acute respiratory distress syndrome, sepsis and septic shock, etc.)) caused by a SARS-CoV-2,

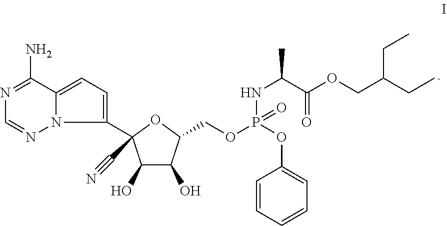 [SMILES: CCC(CC)COC(=O)[C@H](C)NP(=O)(OC[C@H]1O[C@@](C#N)(c2ccc3c(N)ncnn23)[C@H](O)[C@@H]1O)Oc1ccccc1]

The present application also relates to use of the compound represented by Formula I, a geometric isomer, a pharmaceutically acceptable salt, a solvate and/or a hydrate thereof in manufacture of a medicament as a SARS-CoV-2 inhibitor.

The present application also relates to use of the compound represented by Formula I, a geometric isomer, a pharmaceutically acceptable salt, a solvate and/or a hydrate thereof in manufacture of a medicament for inhibiting the replication or reproduction of SARS-CoV-2 in a cell (e.g., a cell of mammal).

The present application also relates to a pharmaceutical composition, which comprises the compound represented by Formula I, a geometric isomer, a pharmaceutically acceptable salt, a solvate and/or a hydrate thereof.

Preferably, the pharmaceutical composition further comprises a pharmaceutically acceptable carrier or excipient. Specifically, the pharmaceutical composition is a solid preparation, an injection, an external preparation, a spray, a liquid preparation, or a compound preparation.

In some embodiments, the pharmaceutical composition comprises an effective amount of the compound represented by formula I, a geometric isomer, a pharmaceutically acceptable salt, a solvate and/or a hydrate thereof.

The present application also relates to use of the pharmaceutical composition comprising the compound represented by Formula I, a geometric isomer, a pharmaceutically acceptable salt, a solvate and/or a hydrate thereof or the compound represented by Formula I, a geometric isomer, a pharmaceutically acceptable salt, a solvate and/or a hydrate thereof in manufacture of a medicament for treating a respiratory disease, including but not limited to simple infections (such as fever, cough and sore throat), pneumonia, acute respiratory infection, severe acute respiratory infection (SARI), hypoxic respiratory failure and acute respiratory distress syndrome, sepsis and septic shock, etc.

The present application also relates to use of the pharmaceutical composition comprising the compound represented by Formula I, a geometric isomer, a pharmaceutically acceptable salt, a solvate and/or a hydrate thereof in manufacture of a medicament for treating a disease or an infection caused by a SARS-CoV-2 (e.g., a respiratory disease (e.g., a simple infection (such as fever, cough and sore throat), pneumonia, acute respiratory infection, severe acute respiratory infection (SARI), hypoxic respiratory failure and acute respiratory distress syndrome, sepsis and septic shock, etc.)).

The present application also relates to use of the pharmaceutical composition comprising the compound of Formula I, a geometric isomer, a pharmaceutically acceptable salt, a solvate and/or a hydrate in manufacture of a medicament as a SARS-CoV-2 inhibitor.

The present application also relates to use of the pharmaceutical composition comprising the compound of Formula I, a geometric isomer, a pharmaceutically acceptable salt, a solvate and/or a hydrate in manufacture of a medicament for inhibiting replication or reproduction of SARS-CoV-2 in a cell (such as a cell of mammal).

The present application also relates to a method for treating and/or preventing a disease in a mammal in need or a method for inhibiting the replication or reproduction of SARS-CoV-2 in a mammal in need, the method comprises administering to the mammal in need a therapeutically and/or prophylactically effective amount of the pharmaceutical composition comprising the compound of Formula I, a geometric isomer, a pharmaceutically acceptable salt, a solvate and/or a hydrate thereof or a compound of Formula I, a geometric isomer, a pharmaceutically acceptable salt, a solvate and/or a hydrate thereof, wherein the disease includes a disease caused by a SARS-CoV-2, such as a viral infectious disease caused by a SARS-CoV-2 (e.g., respiratory disease, including simple infection (such as fever, cough and sore throat, etc.), pneumonia, acute respiratory infection, severe acute respiratory infection (SARI), hypoxic respiratory failure and acute respiratory distress syndrome, sepsis and septic shock, etc.).

The present application also relates to the compound represented by Formula I, a geometric isomer, a pharmaceutically acceptable salt, a solvate and/or a hydrate thereof, for use in treating a disease or an infection (e.g., a respiratory disease (e.g., a simple infection such as fever, cough and sore throat), pneumonia, acute respiratory infection, severe acute respiratory infection (SARI), hypoxic respiratory failure and acute respiratory distress syndrome, sepsis and septic shock, etc.) caused by a SARS-CoV-2.

The present application also relates to the compound represented by Formula I, a geometric isomer, a pharmaceutically acceptable salt, a solvate and/or a hydrate thereof, for use as a SARS-CoV-2 inhibitor.

The present application also relates to the compound represented by Formula I, a geometric isomer, a pharmaceutically acceptable salt, a solvate and/or a hydrate thereof, for use in inhibiting replication or reproduction of SARS-CoV-2 in a cell (e.g., a cell of mammal).

The present application also relates to the pharmaceutical composition comprising the compound represented by Formula I, a geometric isomer, a pharmaceutically acceptable salt, a solvate and/or a hydrate thereof, for use in treating a disease or an infection (e.g., a respiratory disease (e.g., a simple infection (such as fever, cough and sore throat), pneumonia, acute respiratory infection, severe acute respiratory infection (SARI), hypoxic respiratory failure and acute respiratory distress syndrome, sepsis and septic shock, etc.)) caused by a SARS-CoV-2.

The present application also relates to the pharmaceutical composition comprising the compound represented by Formula I, a geometric isomer, a pharmaceutically acceptable salt, a solvate and/or a hydrate thereof, for use as a SARS-CoV-2 inhibitor.

The present application also relates to the pharmaceutical composition comprising the compound represented by Formula I, a geometric isomer, a pharmaceutically acceptable salt, a solvate and/or its hydrate thereof, for use in inhibiting replication or reproduction of SARS-CoV-2 in a cell (e.g., a cell of mammal).

In some embodiments, the disease caused by SARS-CoV-2 described in the present application includes but is not limited to respiratory disease, such as simple infection (such as fever, cough and sore throat), pneumonia, acute respiratory infection, severe acute respiratory infection (SARI), hypoxic respiratory failure and acute respiratory distress syndrome, sepsis and septic shock.

In some embodiments, the disease caused by SARS-CoV-2 described herein is COVID-19.

In the present application, the official name of the term “2019 novel Coronavirus (2019-nCoV)” is severe acute respiratory syndrome coronavirus 2 (SARS-CoV-2).

In the present application, the official name of the term “disease caused by 2019 novel Coronavirus (2019-nCoV)” is COVID-19.

In some embodiments, the mammal includes bovine, equine, caprid, suidae, canine, feline, rodent, primate, wherein the preferred mammal is human, cat, dog or pig.

The pharmaceutical composition described in the present application can be prepared into various forms according to different administration routes.

According to the present application, the pharmaceutical composition can be administered in any one of the following routes: oral administration, spray inhalation, rectal administration, nasal administration, buccal administration, vaginal administration, topical administration, parenteral administration such as subcutaneous, intravenous, intramuscular, intraperitoneal, intrathecal, intraventricular, intrasternal and intracranial injection or infusion, or administration with the help of an explant reservoir, wherein the preferred administration route is oral, intraperitoneal or intravenous.

When orally administered, the compound represented by Formula I, a geometric isomer, a pharmaceutically acceptable salt, a solvate and/or a hydrate thereof can be prepared into any form of orally acceptable preparation, including but not limited to a tablet, a capsule, an aqueous solution or an aqueous suspension. The carrier for use in a tablet generally includes lactose and corn starch, and a lubricant such as magnesium stearate can also be added. The diluent for use in a capsule generally includes lactose and dry corn starch. The aqueous suspension is usually used by mixing an active ingredient with a suitable emulsifier and a suitable suspending agent. If necessary, a sweetener, a flavoring agent or a coloring agent can also be added to the above-mentioned forms of oral preparation.

When rectally administered, the compound represented by Formula I, a geometric isomer, a pharmaceutically acceptable salt, a solvate, and/or a hydrate thereof can generally be prepared in a form of suppository, which is prepared by mixing the drug with a suitable non-irritating excipient. The excipient is present in solid state at room temperature, but melts at the rectal temperature to release the drug. Such excipient includes cocoa butter, beeswax and polyethylene glycol.

When topically administered, especially for treatment of easily accessible affected-surface or organ, such as eye, skin, or lower intestinal neurological disease by topical application, the compound represented by Formula I, a geometric isomer, a pharmaceutically acceptable salt, a solvate and/or a hydrate thereof can be prepared in various forms of topical preparations according to different affected-surfaces or organs, the specific instructions are as follows:

When topically administered to eye, the compound represented by Formula I, a geometric isomer, a pharmaceutically acceptable salt, a solvate and/or a hydrate thereof can be formulated into a preparation form such as micronized suspension or solution, the carrier used is isotonic sterile saline with a certain pH, and a preservative such as benzyl chloride alkoxide may or may not be added. In addition, for administration to eye, the compound can also be prepared in a form of ointment such as vaseline ointment.

When topically administered to skin, the compound represented by Formula I, a geometric isomer, a pharmaceutically acceptable salts, a solvate and/or a hydrate thereof can be prepared into a suitable form such as an ointment, a lotion or a cream, in which the active ingredient is suspended or dissolved in one or more carriers. The carrier for use in an ointment includes, but is not limited to: mineral oil, liquid petrolatum, white petrolatum, propylene glycol, polyethylene oxide, polypropylene oxide, emulsifying wax, and water. The carrier for use in a lotion or a cream includes, but is not limited to: mineral oil, sorbitan monostearate, Tween-60, cetyl ester wax, hexadecenyl aryl alcohol, 2-octyldodecanol, benzyl alcohol and water.

When topically administered to lower intestinal tract, the compound represented by Formula I, a geometric isomer, a pharmaceutically acceptable salt, a solvate and/or a hydrate thereof can be prepared into a form such as rectal suppository as described above or a suitable enema preparation form, in addition, a topical transdermal patch can also be used.

The compound represented by Formula I, a geometric isomer, a pharmaceutically acceptable salt, a solvate and/or a hydrate thereof can also be administered in a preparation form of sterile injection, including sterile injectable aqueous solution or oil suspension, or sterile injectable solutions, wherein the usable carrier and solvent includes water, Ringer's solution and isotonic sodium chloride solution. In addition, a sterilized non-volatile oil such as monoglyceride or diglyceride can also be used as solvent or suspension media.

The drugs of the above various preparation forms can be prepared according to conventional methods in the pharmaceutical field.

In the present application, the term “therapeutically effective amount” or “prophylactically effective amount” refers to an amount that is sufficient to treat or prevent a patient's disease but is sufficiently low to avoid serious side effects (at a reasonable benefit/risk ratio) within a reasonable medical judgment. The therapeutically effective amount of the compound will change according to the factors such as the selected specific compound (e.g., considering the efficacy, effectiveness, and half-life of compound), the selected administration route, the treated disease, the severity of the treated disease, the patient's age, size, weight and physical disease, medical history, duration of treatment, nature of concurrent therapy, desired therapeutic effect, etc., but can still be routinely determined by those skilled in the art.

In addition, it should be noted that the specific dosage and method of using the compound represented by Formula I, a geometric isomer, a pharmaceutically acceptable salt, a solvate and/or a hydrate thereof for different patients depends on many factors, including the patient's age, weight, gender, natural health status, nutritional status, active strength of drug, administration time, metabolic rate, severity of disease, and subjective judgment of physician. Herein it is preferred to use a dosage between 0.001-1000 mg/kg body weight/day.

### BRIEF DESCRIPTION OF THE DRAWINGS

FIG. 1 shows that Remdesivir can effectively reduce the viral nucleic acid load in Vero E6 cells infected by SARS-CoV-2 virus. In FIG. 1, (a) shows that Remdesivir can reduce the viral RNA load in the cells 48 hours after the cells were infected by SARS-CoV-2 virus, and the drug concentration of 33 μM can reduce the viral nucleic acid load by an order of magnitude. The ordinate is the copy number of viral RNA in the sample, and the abscissa is the drug concentration; (b) shows that the test cells were treated by Remdesivir at the test concentration for 48 hours, and no cytotoxicity was observed. The ordinate is the percentage of cell viability relative to the vehicle control group (only cells, no drug added), and the abscissa is the drug concentration.

### SPECIFIC MODELS FOR CARRYING OUT THE APPLICATION

The following examples are illustrative preferred embodiments of the present application and do not constitute any limitation to the present application.

### Example 1: Experiment of Remdesivir in Reduction of Viral Nucleic Acid Load of Cells Infected by SARS-CoV-2 Virus

(1) Drug Treatment of Virus-Infected Cells

Vero E6 cells (purchased from ATCC, Catalog No. 1586) were placed into a 24-well plate and incubated for 24 hours, then virus infection was carried out, specifically, SARS-CoV-2 (2019-nCoV) virus (nCoV-2019BetaCoV/Wuhan/WIV04/2019 strain, provided by Wuhan Institute of Virology, Chinese Academy of Sciences) was diluted with 2% cell maintenance solution (formulation: FBS (purchased from Gibco, Catalog No.: 16000044) was added to MEM (purchased from Gibco, Article No: 10370021) by a volume ratio of 2%, thereby obtaining the 2% cell maintenance solution) to corresponding concentration, and then added to the 24-well plate so that each well contained a viral load of 100TCID₅₀. Next, the Remdesivir (purchased from MedChemExpress, Catalog No.: HY-104077) was diluted with 2% cell maintenance solution to the corresponding concentrations and added to corresponding wells, so that the final drug concentrations were 100 μM, 33 μM, 11 μM, 3.7 μM, 1.23 μM, 0.41 μM, 0.14 μM, respectively, then the plate was put in 37° C., 5% CO₂ incubator and continuously cultured for 48 h; and the vehicle control group was added with only 2% cell maintenance solution without any test drug.

(2) RNA Extraction

The RNA extraction kit was purchased from Qiagen, Catalog No.: 74106. The consumptive materials (spin column, RNase-free 2 ml collection tube, etc.) and reagents (RLT, RW1, RPE, RNase-free water, etc.) involved in the following RNA extraction steps were all parts of the kit.

The following extraction steps were all recommended by the kit instructions.

1) 100 μL of the supernatant was taken from the test plate, added to a nuclease-free EP tube, then added with 350 μL of Buffer RLT, mixed with a transfer liquid gun to make it fully lysed, and centrifuged to take the supernatant;

2) the supernatant obtained in step 1) was added with an equal volume of 70% ethanol and mixed well;

3) the mixed solution obtained in step 2) above was transferred to a RNase-free spin column, centrifuged at 12000 rpm for 15 s, and the waste liquid was discarded;

4) 700 μL of Buffer RW1 was added to the spin column, then centrifugation was carried out at 12000 rpm for 15 s to clean the spin column, and the waste liquid was discarded;

5) 500 μL of Buffer RPE was added to the spin column, then centrifugation was carried out at 12000 rpm for 15 s to clean the spin column, and the waste liquid was discarded;

6) 500 μL of Buffer RPE was added to the spin column, then centrifugation was carried out at 12000 rpm for 2 min to clean the spin column, and the waste liquid was discarded;

7) the spin column was placed in a new RNase-free 2 ml collection tube, and centrifugation was carried out at 12000 rpm for 1 min to dry the spin column, and then the entire spin column was transferred to the 1.5 ml collection tube of step 8);

8) the spin column dried in step 7) was placed in a new 1.5 ml collection tube, added with 30 μl of RNase-free water, and centrifuged at 12000 rpm for 2 min, the obtained eluent contained the corresponding RNA, was added with RNase inhibitor (purchased from NEB, Catalog No.: M0314L), and detected with Nano Drop (purchased from Thermo scientific, Nano Drop One) to determine each RNA concentration.

(3) RNA Reverse Transcription

In the experiment, the reverse transcription kit (PrimeScript™ RT reagent Kit with gDNA Eraser, Catalog No. RR047Q) produced by TaKaRa Company was used for RNA reverse transcription. The steps were as follows.

\textcircled{1} gDNA removal: RNA samples from each experimental group were collected, and 1 μg thereof was taken and subjected to reverse transcription. First, 2 μl of 5×gDNA Eraser Buffer was added to the RNA sample of each experimental group, the reaction system was supplemented with RNase Free water to 10 μl, mixed well, and subjected to 42° C. water bath for 2 min to remove the gDNA that might exist in the sample;

\textcircled{2} Reverse transcription: the sample obtained in \textcircled{1} was added with appropriate amounts of enzyme, primer Mix and reaction buffer, supplemented with RNase Free water to a volume of 20 μl, reacted under 37° C. water bath for 15 min, then put in 85° C. water bath for 5 sec, thereby obtaining cDNA via transcription.

(4) Real-Time PCR

Fluorescence quantitative PCR was used to detect the copy number per ml of the original virus solution.

The reaction system was mixed using TB Green Premix (Takara, Cat #RR820A), and the amplification reaction and reading were carried out with StepOne Plus Real-time PCR instrument (brand: ABI). The copy number contained in per ml of the original virus solution was calculated. The steps were as follows:

\textcircled{1} Establishment of standard product: the plasmid pMT-RBD (the plasmid was provided by Wuhan Institute of Virology, Chinese Academy of Sciences) was diluted to 5×10⁸ copies/μL, 5×10⁷ copies/μL, 5×10⁶ copies/μL, 5×10⁵ copies/μL, 5×10⁴ copies/μL, 5×10³ copies/μL, 5×10² copies/μL. 2 μL standard or cDNA template was taken for qPCR reaction.

\textcircled{2} The sequences of primers used in the experiment were as follows (all indicated in 5′-3′ direction):

RBD-qF:
CAATGGTTTAACAGGCACAGG
RBD-qR:
CTCAAGTGTCTGTGGATCACG

\textcircled{1} The reaction procedure was as follows:

Pre-denaturation: 95° C. for 5 minutes;

Cycle parameters: 95° C. for 15 seconds, 54° C. for 15 seconds, 72° C. for 30 seconds, for a total of 40 cycles.

(5) Cytotoxicity Test of Drug

The detection of the drug cytotoxicity was performed using CCK-8 kit (Beoytime). Specific steps were as follows:

\textcircled{1} 1×10⁴ Vero E6 (ATCC) cells were placed in a 96-well plate and incubated at 37° C. for 8 hours.

\textcircled{2} The drug was diluted with DMSO to an appropriate concentration of mother liquor, and then diluted with MEM medium (purchased from Gibco, Catalog No. 10370021) containing 2% FBS (purchased from Gibco, Catalog No. 16000044) to the same concentration as that for the drug treatment. The original medium in the 96-well plate was discarded, 100 μL of drug-containing MEM medium was added to the cells, and three replicate wells were prepared for each concentration. Vehicle control (DMSO and medium were added to the cell wells, without adding drug) and blank control (DMSO and medium were added to the wells, without cells) were set up. After the drug was added, the cells were cultured at 37° C. for 48 hours.

\textcircled{3} 20 μL of CCK-8 solution (Beoytime) was added to the well to be tested, mixed gently, without generating bubbles, and continuously incubated at 37° C. for 2 hours. OD₄₅₀ was read on a microplate reader (purchased from Molecular Devices, Model: SpectraMax M5), and cell viability was calculated:

Cell activity (%)=(A_{(drug treatment group)}−A_{(blank control)})/(A_{(vehicle control)}−A_{(blank control)})×100%

wherein A was the reading of the microplate reader.

(6) Experimental Results

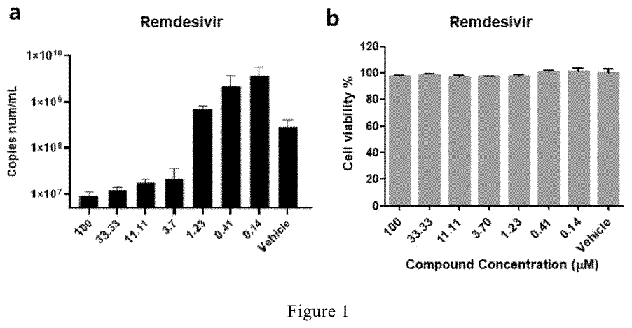

The results of the virus proliferation inhibition experiment showed that the test compound at concentrations of 100 μM, 33 μM, 11.1 μM and 3.7 μM could effectively inhibit the replication of the SARS-CoV-2 virus genome in the infected supernatant (Table 1 and FIG. 1)

TABLE 1
In vitro antiviral test of the test compound (Remdesivir)

| | Concentration (μM) | | | | | | | |
|---|---|---|---|---|---|---|---|---|
| | 100 | 33.33 | 11.11 | 3.70 | 1.23 | 0.41 | 0.14 | Vehicle |
| Virus genome | 9010444.5 ± | 12429732 ± | 16308516.5 ± | 20287512.5 ± | 879273650.5 ± | 1751863138.5 ± | 6120820311.5 ± | 510815026 ± |
| copy number | 2282239.02 | 1812581.22 | 1475181.58 | 13084251.56 | 189609425.32 | 1115069812.46 | 2886728551.13 | 180518414.22 |

The cytotoxicity test results showed that the treatment of the test compound (Remdesivir) did not change the cell viability at all test concentrations, that was, the test compound had no toxic effect on the cells at all concentrations (Table 2 and FIG. 1).

TABLE 2
Cytotoxicity test results of the test compound (Remdesivir)

| | Concentration (μM) | | | | | | | |
|---|---|---|---|---|---|---|---|---|
| | 100 | 33.33 | 11.11 | 3.70 | 1.23 | 0.41 | 0.14 | Vehicle |
| Cell viability | 97.70 ± | 99.06 ± | 97.05 ± | 97.40 ± | 97.88 ± | 100.76± | 100.97 ± | 100.19 ± |
| (% of vehicle | 0.76 | 0.41 | 1.12 | 0.41 | 1.12 | 1.17 | 2.61 | 2.62 |
| control) | | | | | | | | |

What is claimed is:

1. A method for treating and/or preventing a disease in a mammal in need, the method comprising administering to the mammal in need a therapeutically and/or prophylactically effective amount of a pharmaceutical composition comprising a compound represented by Formula I, a geometric isomer, a pharmaceutically acceptable salt, a solvate and/or a hydrate thereof, or a compound represented by Formula I, a geometric isomer, a pharmaceutically acceptable salt, a solvate and/or a hydrate thereof, 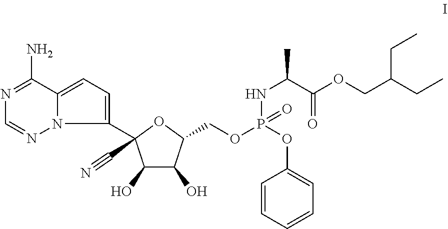 [SMILES: CCC(CC)COC(=O)[C@H](C)NP(=O)(OC[C@H]1O[C@@](C#N)(c2ccc3c(N)ncnn23)[C@H](O)[C@@H]1O)Oc1ccccc1]
wherein the disease is a disease caused by a SARS-CoV-2.

2. The method according to claim 1, wherein the disease caused by a SARS-CoV-2 is a viral infectious disease caused by a SARS-CoV-2.

3. The method according to claim 1, wherein the disease caused by a SARS-CoV-2 is a respiratory disease.

4. The method according to claim 1, wherein the disease caused by a SARS-CoV-2 is simple infection, pneumonia, acute respiratory tract infection, severe acute respiratory infection (SARI), hypoxic respiratory failure, acute respiratory distress syndrome, sepsis or septic shock.

5. The method according to claim 4, wherein the simple infection is fever, cough and/or sore throat.

6. The method according to claim 1, wherein the pharmaceutical composition further comprises a pharmaceutically acceptable carrier or excipient.

7. The method according to claim 6, wherein the pharmaceutical composition is a solid preparation, an injection, an external preparation, a spray, a liquid preparation, or a compound preparation.

8. The method according to claim 1, wherein the mammal is bovine, equine, caprid, suidae, canine, feline, rodent or primate.

9. The method according to claim 8, wherein the mammal is human, cat, dog or pig.

10. A method for inhibiting the replication or reproduction of SARS-CoV-2 in a mammal in need, the method comprising administering to the mammal in need a therapeutic and/or prophylactically effective amount of a pharmaceutical composition comprising a compound represented by Formula I, a geometric isomer, a pharmaceutically acceptable salt, a solvate and/or a hydrate thereof, or a compound represented by Formula I, a geometric isomer, a pharmaceutically acceptable salt, a solvate and/or a hydrate,

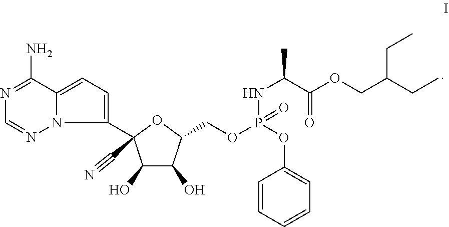 [SMILES: CCC(CC)COC(=O)[C@H](C)NP(=O)(OC[C@H]1O[C@@](C#N)(c2ccc3c(N)ncnn23)[C@H](O)[C@@H]1O)Oc1ccccc1]

11. The method according to claim 10, wherein the pharmaceutical composition further comprises a pharmaceutically acceptable carrier or excipient.

12. The method according to claim 11, wherein the pharmaceutical composition is a solid preparation, an injection, an external preparation, a spray, a liquid preparation, or a compound preparation.

13. The method according to claim 1, wherein the disease caused by a SARS-CoV-2 is COVID-19.

14. The method according to claim 8, wherein the mammal is bovine, equine, caprid, suidae, canine, feline, rodent, primate.

15. The method according to claim 14, wherein the mammal is human, cat, dog or pig.
